(19) United States
(12) Patent Application Publication
(10) Pub. No.: US 2025/0073036 A1
(43) Pub. Date: Mar. 6, 2025

(54) BONE PLUG

(71) Applicant: Zimmer, Inc., Warsaw, IN (US)

(72) Inventors: George Andrews, Farmington, CT (US); Christopher Westbrook, Shelton, CT (US); Marton Varady, Farmington, CT (US); Maxim Budyansky, Baltimore, MD (US); Neil Shah, Baltimore, MD (US)

(21) Appl. No.: 18/819,692

(22) Filed: Aug. 29, 2024

Related U.S. Application Data
(60) Provisional application No. 63/536,241, filed on Sep. 1, 2023.
(60) Provisional application No. 63/539,162, filed on Sep. 19, 2023.

(51) Int. Cl. A61F 2/28

(52) U.S. Cl. CPC A61F 2/28; A61F 2002/2839; A61F 2220/0016; A61F 2230/0086; A61F 2250/0024

(57) ABSTRACT

Methods and devices for plugging a hole bone are disclosed. The bone plug device may include a polymeric mesh structure and a first and a second solid polymeric structure mounted or formed onto a first and a second corresponding leaflet of the polymeric mesh structure. When inserted into a hole in cortical bone, the polymeric mesh structure moves from an open configuration to a closed configuration, reducing the diameter of the polymeric mesh structure and causing the first solid polymeric structure to contact the second solid polymeric structure, conforming bone plug device to the hole. The polymeric mesh structure is configured to mimic cancellous bone structure and porosity to encourage cancellous bone ingrowth.

### CLAIM OF PRIORITY

This application claims the benefit of U.S. Provisional Patent Application Ser. No. 63/536,241, filed on Sep. 1, 2023, and also claims the benefit of U.S. Provisional Patent Application Ser. No. 63/539,162, filed on Sep. 19, 2023, the benefit of priority of each of which is claimed hereby, and each of which is incorporated by reference herein in its entirety.

### TECHNICAL FIELD

Methods and devices for plugging holes in cortical bone are disclosed.

### BACKGROUND

Bone grafts are used in surgical procedures that require the fusion, healing or joining of bones. Autologous bone grafts are harvested from a patient's own body, for example by creating a hole in the cortex to access the underlying cancellous bone. Such holes may be plugged to facilitate healing of the holes.

### BRIEF DESCRIPTION OF THE DRAWINGS

FIG. 1A schematically shows a perspective view of a variable diameter bone plug device according to the present disclosure.

FIG. 1B schematically shows a cross-section view of the bone plug device shown in FIG. 1A.

FIG. 2A schematically shows a perspective view of a bone plug device including a stem according to the present disclosure.

FIG. 2B schematically shows a cross-section view of the bone plug device including a stem shown in FIG. 2A.

FIG. 3A schematically shows a top view of a bone plug device including a stem in an open configuration.

FIG. 3B schematically shows a top view of the bone plug device shown in FIG. 3A in a closed configuration.

FIG. 4A schematically shows a perspective view of a bone plug device including thread retention features according to the present disclosure.

FIG. 4B schematically shows a side view of the bone plug device including thread retention features shown in FIG. 4A.

FIG. 5 schematically shows three views of a “top hat” configuration of a bone plug device according to the present disclosure.

FIG. 6 schematically shows views of a bone plug device including a solid rail structure according to the present disclosure.

FIG. 7 schematically shows a perspective view of a bone plug device including retention features according to the present disclosure.

FIG. 8 schematically shows a cross-section view of a bone plug device including a cannulation feature according to the present disclosure.

FIG. 9 schematically shows a cross-section view of a bone plug device including a solid core and leaf framework according to the present disclosure.

FIG. 10A schematically shows alignment of a bone plug device with a cortical hole according to the present disclosure.

FIG. 10B schematically shows initial insertion of a bone plug device with a cortical hole.

FIG. 10C schematically shows compression of a fully inserted bone plug device within a cortical hole.

FIG. 10D schematically shows bone ingrowth into a bone plug device within a cortical hole.

FIG. 11A schematically shows a front view of a bone plug device with a flexible region including a series of rings according to the present disclosure.

FIG. 11B schematically shows a close-up cross-section view of the bone plug device shown in FIG. 11A.

FIG. 11C schematically shows a side view of the bone plug device shown in FIG. 11A.

FIG. 11D schematically shows another side view of the bone plug device shown in FIG. 11A.

FIG. 11E schematically shows a top view of the bone plug device shown in FIG. 11A.

FIG. 12A schematically shows a perspective view of a bone plug device with solid rims and a flexible region including a series of rings according to the present disclosure.

FIG. 12B schematically shows a top view of the bone plug device shown in FIG. 12A.

FIG. 12C schematically shows a front view of the bone plug device shown in FIG. 12A.

FIG. 12D schematically shows a cross-section view of the bone plug device shown in FIG. 12A.

FIG. 12E schematically shows a side view of the bone plug device shown in FIG. 12A.

FIG. 12F schematically shows a close-up view of the bone plug device shown in FIG. 12E.

FIG. 13A schematically shows a front view of a bone plug device with a tapered scaffold structure and interconnected pores according to the present disclosure.

FIG. 13B schematically shows a cross-section view of the bone plug device shown in FIG. 13A.

FIG. 13C schematically shows a perspective view of the bone plug device shown in FIG. 13A.

FIG. 14 schematically shows a perspective view of a tapered bone plug device according to the present disclosure.

FIG. 15 schematically shows a perspective view of a three leaflet feature of a bone plug device according to the present disclosure.

### DETAILED DESCRIPTION

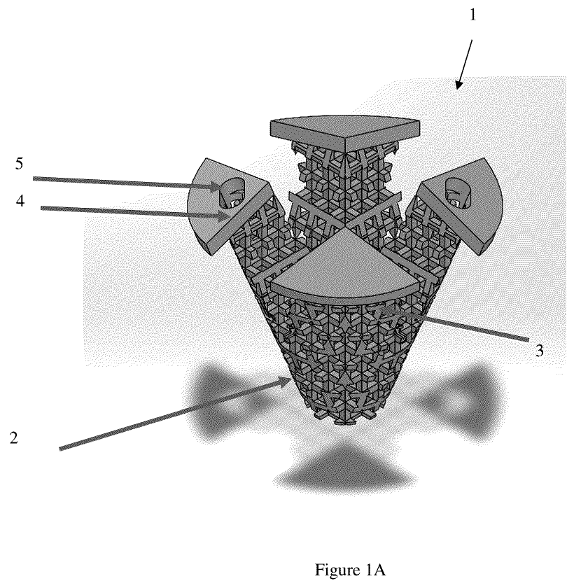

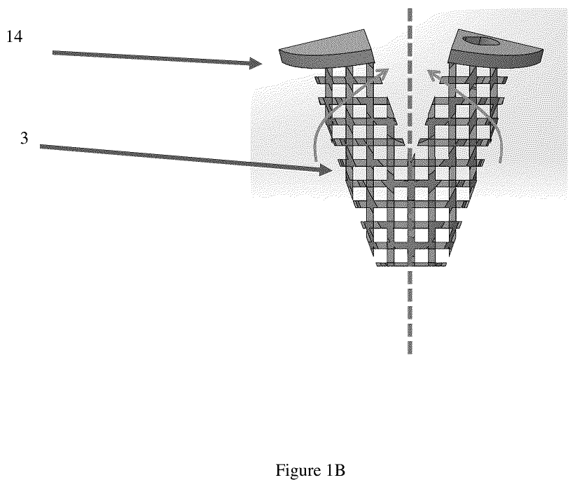

FIG. 1A schematically shows a perspective view of a variable diameter bone plug device 1 for plugging a hole in cortical bone according to the present disclosure. The bone plug device includes a mesh structure 2 that defines a proximal end and a distal end of the device 1. The mesh structure 2 may include at least a first leaflet and a second leaflet 3 joined at the distal end. The mesh structure 2 may be configured to mimic cancellous bone structure and porosity to encourage cancellous bone regeneration and ingrowth into the mesh structure.

In some embodiments, the mesh structure 2 is tapered. A tapered mesh structure 2 allows for deeper insertion into a cortical hole having a variable diameter as well as enabling closure of cortical holes of variable sizes. The shape of the individual mesh cells within the mesh structure 2 may be chosen from various shapes, including but not limited to diamonds, spheres, pyramids, circles, and triangles.

The bone plug device 1 further includes at least one solid structure 4 mounted or formed with respect to a proximal end of a leaflet 3. In some embodiments, each leaflet 3 is paired with a solid structure 4. The one or more solid structures 4 may be configured to mimic cortical bone structure and encourage cortical bone ingrowth into the solid structure. The at least one solid structure 4 may be composed of resorbable or non-resorbable polymers or other biocompatible materials. Biocompatible materials may include minerals, ceramics, or metals that are not expected to trigger an inflammatory or immune response in an implantation site. The solid structure 4 may further include at least one hole 5 configured to receive an insertion tool. The insertion tool may be used by surgeon to grasp the bone plug device 1 and insert the plug device 1 into a hole in a cortical bone. The insertion tool may deploy the plug device 1 into the cortical hole with the leaflets 3 in a compressed or in a closed state such that when the insertion tool is removed, the leaflets 3 spring radially outward to lock into place.

FIG. 1B schematically shows a cross-section view of bone plug device 1 for plugging a hole in cortical bone according to the present disclosure. In some embodiments, the mesh structure 2 is moveable between an open configuration in which the mesh structure 2 defines a first diameter and in which solid structures 4 are spaced from one another, and a closed configuration in which the mesh structure 2 defines a second diameter smaller than the first diameter and in which the solid structures 4 are in contact with one another. For example, in an open configuration the mesh structure 2 defines a diameter of 15 millimeters and in a closed configuration the mesh structure 2 defines a diameter of 10 millimeters. In this specific example, the mesh structure 2 can plug hole sizes ranging between and including a hole size of 10 mm to 15 mm. The dashed line in FIG. 1B depicts where the leaflets 3 of the mesh structure 2 meet in a closed configuration. In some embodiments, when the solid structures 4 are in contact in a closed configuration, the solid structures 4 form a flat rim at the top of the cortical hole. The flat rim configuration reduces detectability of the bone plug device 1 subcutaneously to the patient. The rim further provides a surface to catch onto the surrounding cortex of the hole, preventing the bone plug device 1 from being pushed through the cortical hole. The rim further serves as a cap to the hole to reduce seepage of blood and fluids from permeating through the cortical hole. The plug can allow for air permeability to alleviate intraosseous pressure from void fillers or the perfusion of blood into the underlying cancellous space. A mechanical barrier such as the bone plug device 1 can reduce risk of hematoma when used in a cortical hole with an underlying void.

When implanted into a hole, the bone plug device 1 provides mechanical hemostasis to the device by preventing blood from exiting the hole. For example, the flat rim configuration may sufficiently fill the top of the cortical hole, preventing blood from emerging from the hole. Further, the leaflets 3 impart a spring-like force on the bone material of the inner wall of the cortical bone, assisting in retention and spurring ingrowth of bone through radial force.

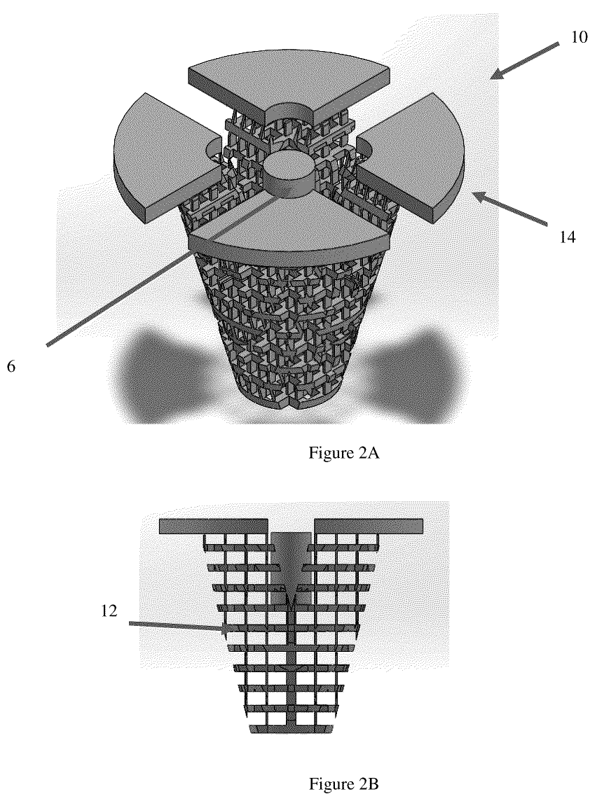

FIG. 2A schematically shows a perspective view of a bone plug device 10 including mesh structure 12 and a stem 6 according to the present disclosure. The stem 6 could be used to grab the device 10 with tweezers, or graspers. In some embodiments, the stem 6 may be cylindrical. In other embodiments, the stem 6 could be a square peg to enable a “socket wrench” insert adaptable over the stem 6 and allow a user to rotate the device 10 into a hole or manipulate the device 10 until the device 10 sits flush with the cortical layer of a bone site. The stem 6 may be configured to increase the strength of the device 10 and to decrease the likelihood of the device 10 breaking during implantation.

Embodiments including a stem 6 may lack insertion tool holes 5 in the solid structures 14 (as is/are included in device 1). The stem 6 may be composed of a polymeric material or other biocompatible material. The stem 6 may be composed of the same material as the mesh structure 12 or the solid structure 14. FIG. 2B schematically shows a cross-section view of the bone plug device 10 including a stem 6 as shown in FIG. 2A.

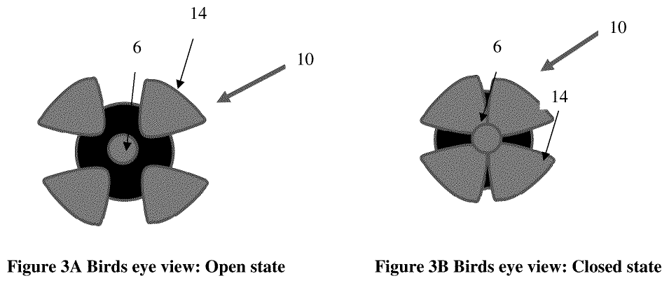

FIG. 3A schematically shows a top view of bone plug device 10 in an open configuration. As discussed above, in an open configuration the solid structures 14 are spaced from one another. In the embodiment depicted in FIG. 3A, a stem 6 is situated in the center of the mesh structure 12 and is spaced from the solid structures 14 in the open configuration.

FIG. 3B schematically shows a top view of the bone plug device 10 shown in FIG. 3A in a closed configuration. As discussed above, in a closed configuration, the solid structures 14 are in contact with one another (or in close juxtaposition). In embodiments including a stem 6, the solid structures 14 may contact the stem 6 in a closed configuration (or be in close juxtaposition).

The solid structures 14 may be configured to ensure the device 10 does not fall through the anatomical hole when in a closed configuration. The solid structures 14 may also be configured to allow the device 10 to be compressed while being pushed into the hole. In some embodiments, the solid structures 14 are configured such that when the device 10 is fully compressed, the solid structures 14 creates a circle or a “cover” to cover the anatomical hole completely.

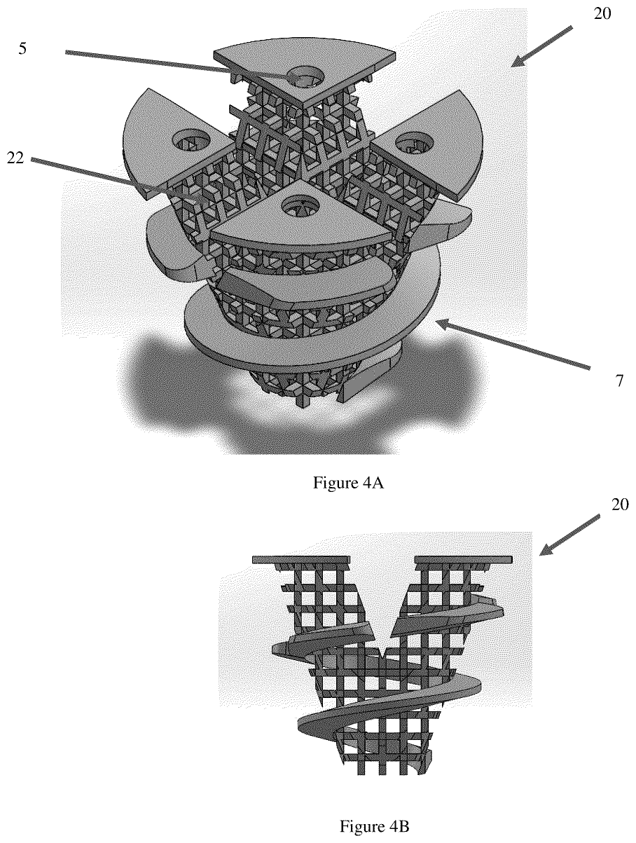

FIG. 4A schematically shows a perspective view of a bone plug device 20 including thread retention feature(s) 7. In some embodiments, the thread retention feature(s) 7 are one or more threads formed along the tapered body of the mesh structure 22. The threads allow a user to utilize a screw action to insert the device 20 while also providing retention forces once the mesh structure 22 is fully inserted in the anatomical hole. The thread retention feature(s) 7 may be paired with a stem and/or insertion tool holes 5 to aid insertion of the device 20.

FIG. 4B schematically shows a side view of the bone plug device 20 including thread retention features 7 shown in FIG. 4A. In some embodiments, the thread retention features 7 may constitute interrupted threads that align in a helical manner. In other embodiments, the thread retention features 7 may constitute a continuous thread, e.g., defining a helical geometry. For example, the thread retention features 7 may constitute half-moon sickles that protrude outwards from the tapered mesh structure 22. The thread retention features 7 provide additional mechanical hemostasis to the implanted device and increase the pull-out strength of the bone plug device 20.

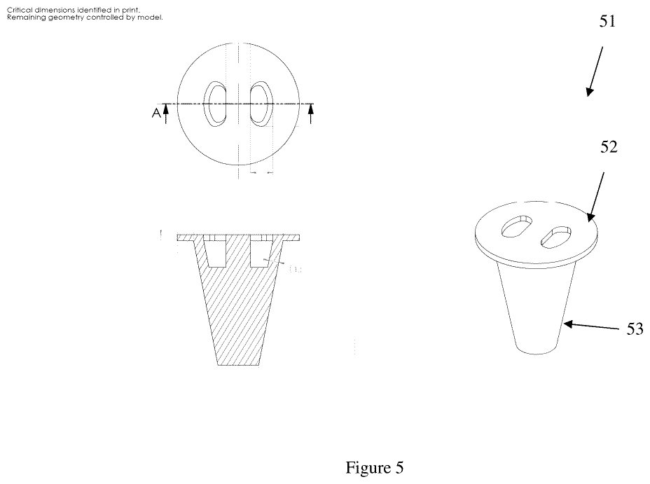

FIG. 5 schematically shows three views of a “top hat” configuration of a bone plug device 51 according to the present disclosure. Bone plug device 51 may be produced in one of various sizes to accommodate varying diameters of cortical/anatomical holes. A user may measure the size of the hole to be plugged via a pin gauge or other suitable means and then select an appropriately sized bone plug device 51. Bone plug device 51 may include a solid “cortical” layer material to compose the rim 52 and a cancellous bone-like material for the tapered body 53. The cancellous bone-like material be configured to have the compressibility of a cork that can conform to multiple diameters under a press fit insertion. Embodiments of bone plug device 51 may include a stem, insertion tool hole(s), or a combination thereof. FIG. 5 includes exemplary dimensions of a bone plug device 51, but the size of the bone plug device 51 is not limited to the dimensions set forth therein.

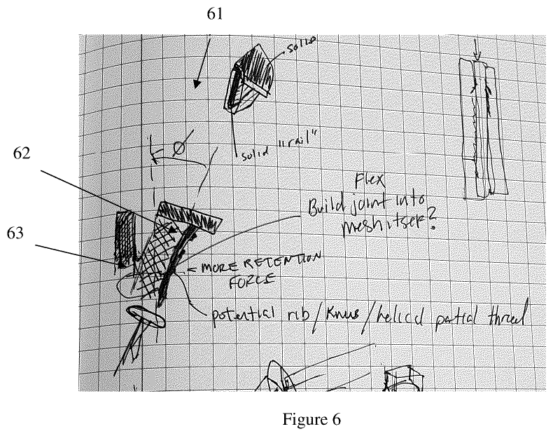

FIG. 6 schematically shows views of a bone plug device 61 including a solid rail structure 62. The solid rail structure 62 may be a rail of solid material running down a leaflet 63. The solid rail structure 62 may be configured to provide additional strength and improve retention of the bone plug device 61 in a cortical/anatomical hole. The solid rail structure 62 may be composed of the same material as the mesh structure or the solid structures of the device 61.

FIG. 7 schematically shows a perspective view of a bone plug device 71 including retention features 74 according to the present disclosure. The retention features 74 may be, for example, a thread, a protrusion, a pin, thorn, barb, or other structure suitable for retaining the bone plug device 71 into a cortical hole. For example, threads or thorns may be situated on an outer surface of the bone plug device 71 and configured to dig into surrounding bone. In some embodiments, the retention features 74 may be configured to provide an audible “click” when the bone plug device 71 is set into an anatomical hole to provide tactile feedback to the user.

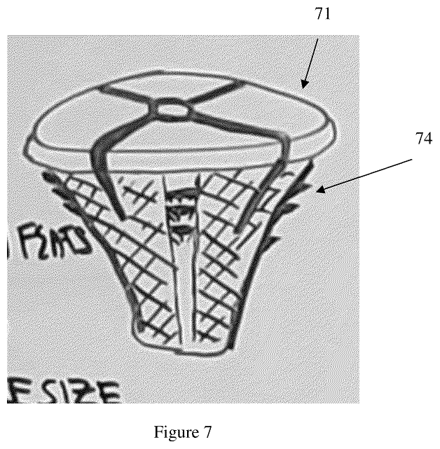

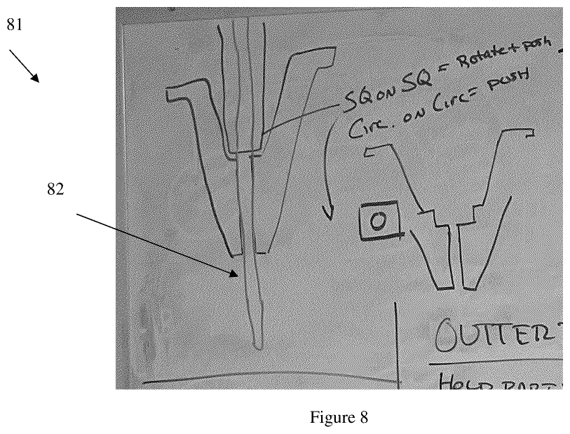

FIG. 8 schematically shows a cross-section of a bone plug device 81 including a cannulation feature 82 according to the present disclosure. The cannulation feature 82 allows guidewire assisted placement of the bone plug device 81. The cannulation feature 82 may be paired with an insertion tool that threads over the guidewire to interface with the bone plug device 81. The insertion tool can communicate via push, torque, or tap onto the bone plug device 81 to insert the device 81 into a cortical/anatomical hole. The insertion tool may have an extension that defines a male geometry, such as a triangular or polygonal shape, that is configured and dimensioned to interface with a female negative slot on the bone plug device 81 to enable rotational actuation of the device 81. The male/female geometric features may be reversed, as will be readily apparent to persons skilled in the art. The slot can be configured to allow rotational insertion of the bone plug device 81, push or tapping insertion of the bone plug device 81, or a combination of both.

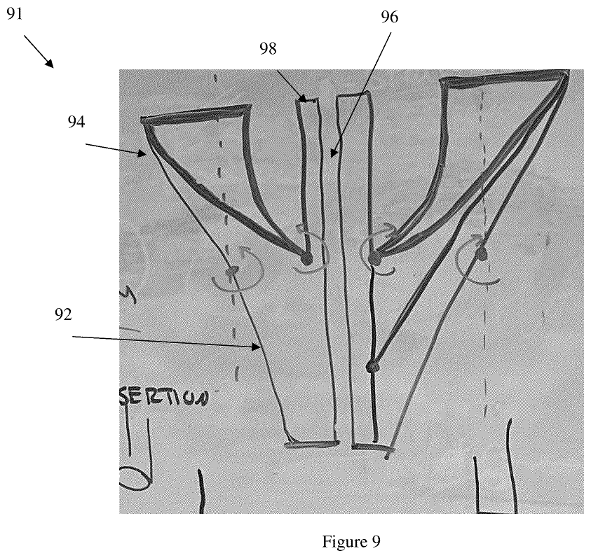

FIG. 9 schematically shows a cross-section view of a bone plug device 91 including a solid core 98 and leaf framework according to the present disclosure. Solid core 98 may extend from the solid structures 94 and connect to stem 96 or a central core region of the bone plug device 91. The solid core 98 be a solid branch of material embedded in the mesh structure 92 that is configured to increase the flex force and retention of the bone plug device 91. The solid core 98 may function as a connection point for a framework of branches that can be oriented to enhance the spring force of the leaflets of the mesh structure 92. The spaces in between the solid framework can be filled with mesh pores for bone ingrowth surfaces. FIG. 9 depicts one embodiment of solid branches of a core 98 that may be incorporated into the bone plug device 91 to increase structural integrity of the device 91.

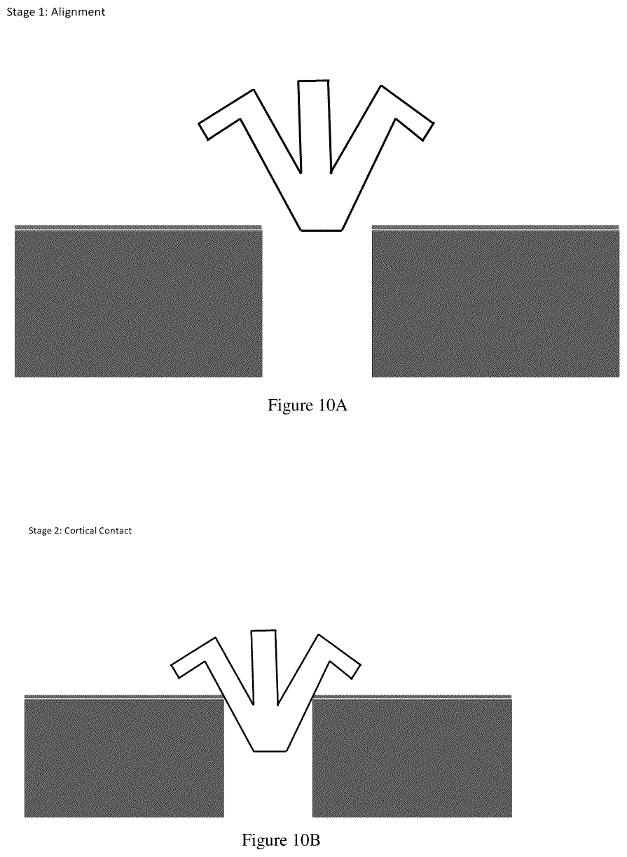

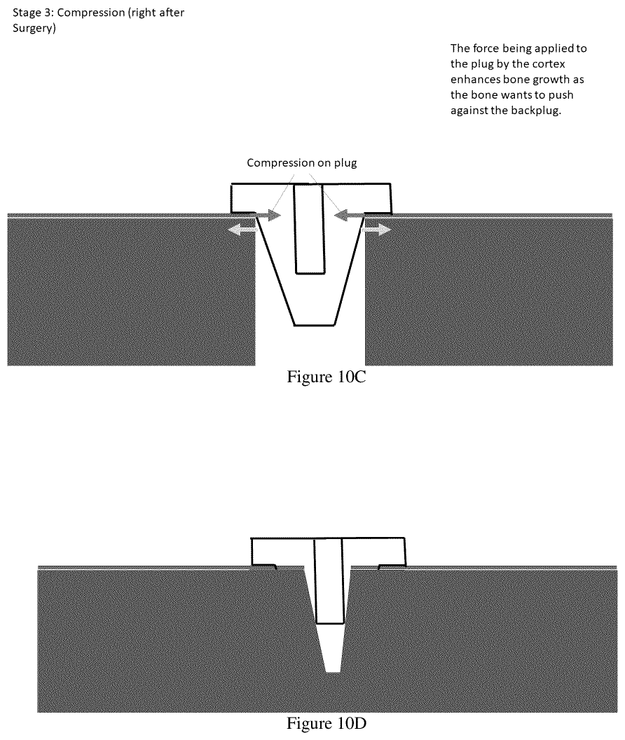

FIGS. 10A-10D depict steps of a method of inserting a bone plug device into a cortical bone hole. FIG. 10A schematically shows alignment of a bone plug device with a cortical hole. A user may insert the bone plug device into an incision, i.e. hole, in a cortical bone and thread the tapered end of the bone plug device into the cortical bone hole. The bone plug device may initially be in an open configuration.

FIG. 10B schematically shows initial insertion of a bone plug device with a hole. The bone plug device may be pushed into place, or forcibly rotated into place within the cortical/anatomical hole. As the bone plug device is inserted, the leaflets of the bone plug device begin to compress inwards, moving from an open configuration to a closed configuration.

FIG. 10C schematically shows compression of a fully inserted bone plug device within a cortical/anatomical hole. When completely seated in the cortical/anatomical hole, the bone plug device generates contact forces circumferentially against the surrounding bone material. The surrounding bone material provides a counter force compressing the bone plug device. These active forces provide mechanical stimulation at the interface between the bone plug device and surrounding bone material, anchoring the bone plug device in place and spurring bone growth. The bone plug device may include leaflets that, in a completely closed state, accommodate the plugging of a minimum diameter hole. The leaflets can form retention across a variety of diameters. The range of the diameters that this variable diameter plug can fill are determined by the open and closed state diameters of the plug. The plug can retain any diameters within this range when fully seated into the hole.

FIG. 10D schematically shows bone growth into a bone plug device within an anatomical hole according to the present disclosure. After implantation, cancellous bone cells will ingrow through the mesh structure of the bone plug device, and the solid structures of the device will similarly integrate with cortical bone over time. The implanted bone plug device is configured to enable closure of the cortical/anatomical hole over time by allowing integration of native bone. In some embodiments, eventually no bone plug device material remains in the anatomy/bone once fully resorbed. If the base material is non-resorbable, then bone will ingrow through the mesh structure.

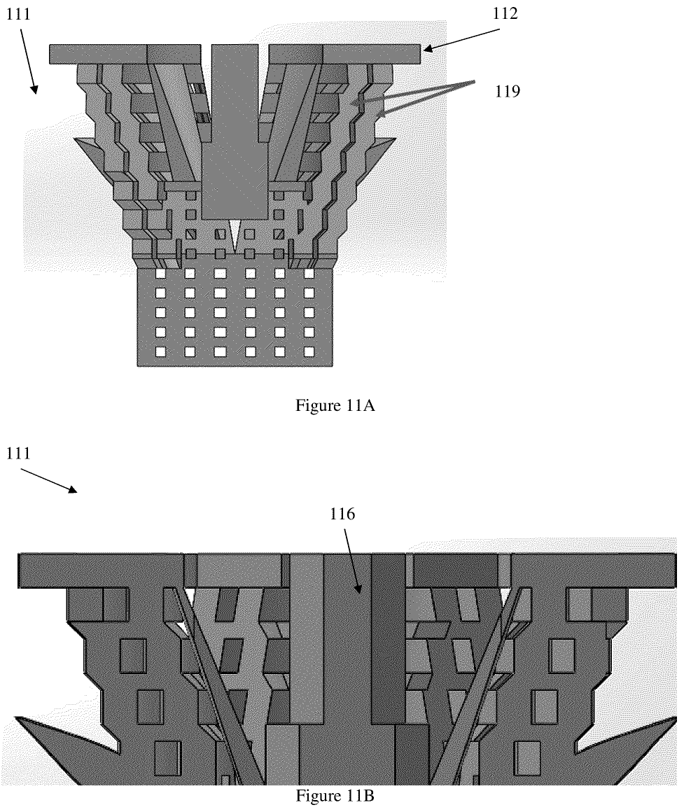

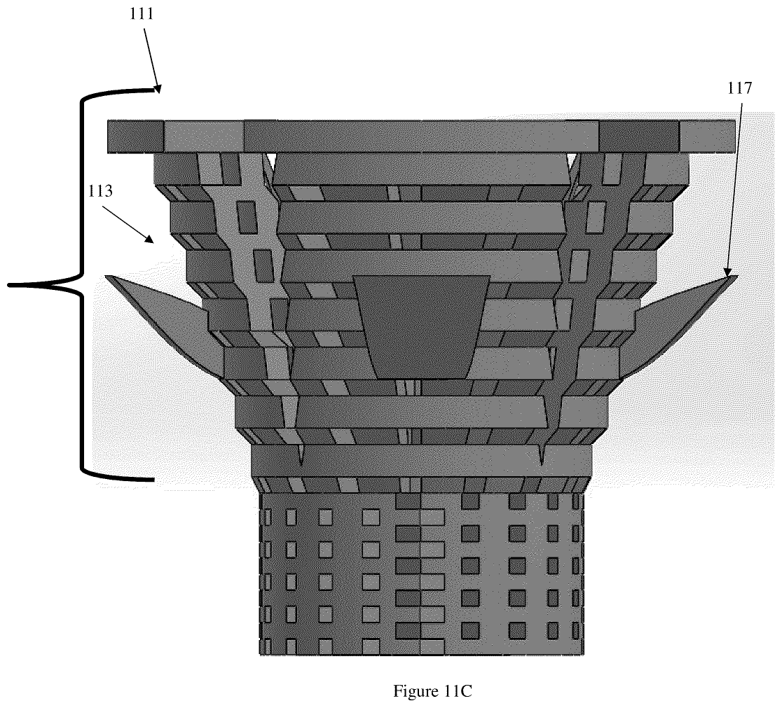

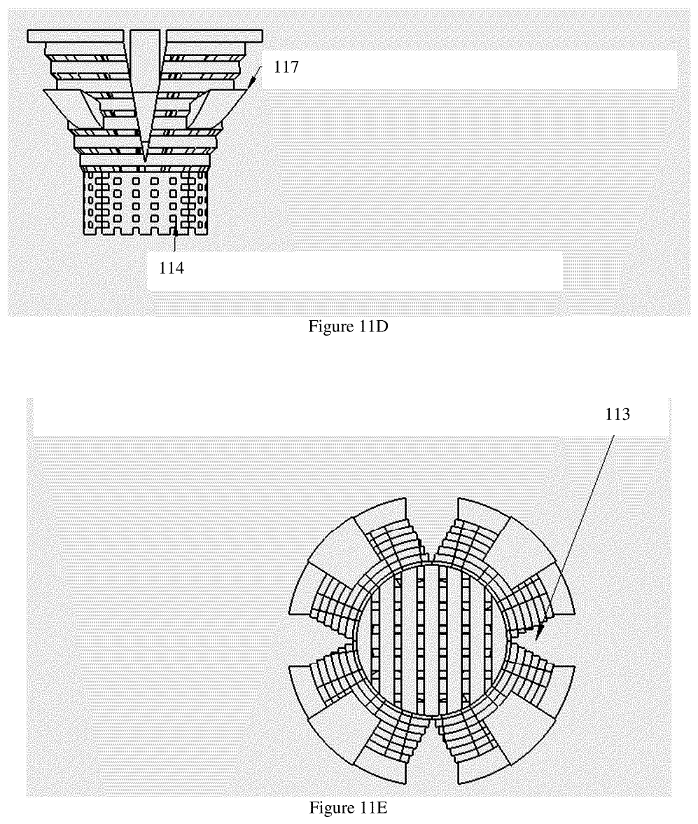

FIG. 11A schematically shows a front view of a bone plug device 111 with a flexible region including a series of rings 119 according to the present disclosure. The flexible region of the plug device 111 may be made up of a series of rings 119 that get larger in diameter as the rings 119 reach the rims 112 of the plug device 111 Each ring 119 may be separated by a gap connected by a circular pattern of pillars spaced equally apart creating a single porous layer on the outermost portion of the plug device 111. Each ring 119 may also contain another, smaller, ring that creates a second layer of pores. The described layers create interconnectivity throughout the pores to allow nutrient flow though the cancellous region of the plug device 111.

FIG. 11B schematically shows a close-up cross-section view of the bone plug device 111 shown in FIG. 11A. This cross-section depicts the stem 116 that may be used to easily attach the device 111 to an insertion tool to facilitate easy deployment of device 111. Stem 116 may be structured similarly to the stem 6 discussed in further detail above.

FIG. 11C schematically shows a side view of the bone plug device 111 shown in FIG. 11A. FIG. 11C shows the flexible, yet stiff, retention features 117 that protrude from the leaves 113 of the device 111. Retention features 117 may be structurally similar to the retention features 7 discussed in further detail above.

FIG. 11D schematically shows another side view of the bone plug device 111 shown in FIG. 11A. The retention features 117 may be configured to secure the plug device 111 in an implantation site, such as a cortical defect, and cause the plug device 111 to be very difficult to dislodge from the cortical defect. In some embodiments, the retention features 117 are not included on the porous structure 114.

FIG. 11E schematically shows a top view of the bone plug device 111 shown in FIG. 11A. FIG. 11E shows flexible leaves 113 configured to make the device 111 useable in a variety of hole sizes and employable when a cortical defect is oblong in shape. For example, the flexible leaves 113 may be compressed inwardly to a desired diameter to fit within a cortical defect.

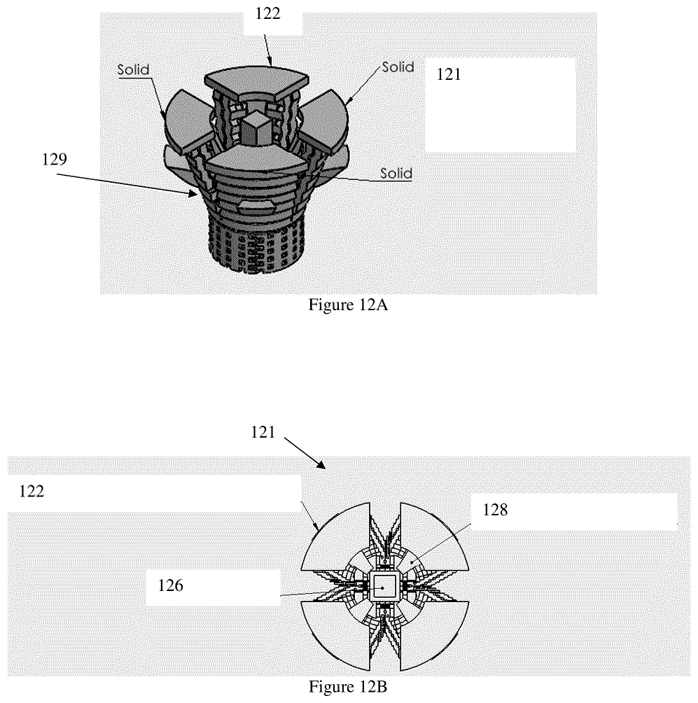

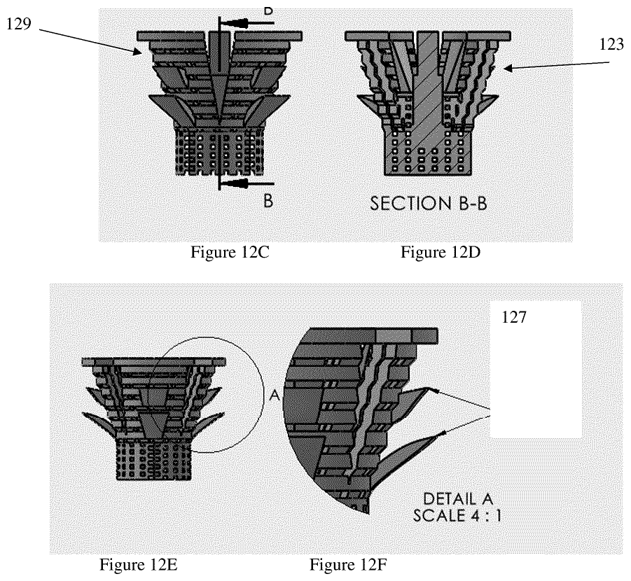

FIG. 12A schematically shows a perspective view of a bone plug device 121 with solid rims 122 and a flexible region including a series of rings 129 according to the present disclosure. In some embodiments, the proximal end of the device 121 includes solid rims 122 configured to prevent the device 121 from falling through a cortical window. Beneath the rims 122 is a porous structure compatible with the cancellous region of bone.

FIG. 12B schematically shows a top view of the bone plug device 121 shown in FIG. 12A. In some embodiments, pillars 128 may be incorporated in the plug device 121 to increase the stiffness of an otherwise very flexible porous structure 124. The pillars 128 help retain the plug device 121 inside the cortical window by producing higher radial forces on the cortical edges. The pillars 128 may be included in embodiments of the device that further include a stem 126.

FIG. 12C schematically shows a front view of the bone plug device 121 shown in FIG. 12A. FIG. 12D schematically shows a cross-section view of the bone plug device 121 shown in FIG. 12A. The cross-section view shows the gradient-style ring design in the leaves 123 similar to the rings 129 discussed with regards to device 121. In some embodiments, the distal end of the device 121 includes porous scaffolding.

FIG. 12E schematically shows a side view of the bone plug device 121 shown in FIG. 12A. FIG. 12F schematically shows a close-up view of the bone plug device 121 shown in FIG. 12E. In some embodiments, the plug device 121 includes one or more retention fins 127, for example two retention fins. The retention fins 127 may be configured to aid in the retention of the device 121 within a cortical defect. The retention fins 127 may be configured to increase the amount of force/pressure needed for the device 121 to become dislodged from a cortical window.

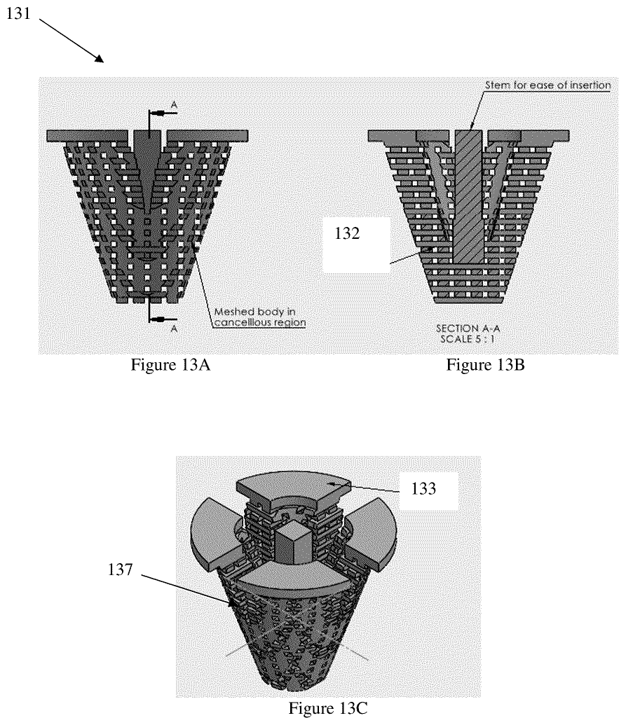

FIG. 13A schematically shows a front view of a bone plug device 131 with a tapered scaffold structure 132 and interconnected pores according to the present disclosure. FIG. 13B schematically shows a cross-section view of the bone plug device 131 shown in FIG. 13A. In some embodiments, all of the pores of the tapered scaffold structure 132 are interconnected. In some embodiments, substantially all of the pores of the tapered scaffold structure 132 are interconnected, for example 90%, 91%, 92%, 93%, 94%, 95%, 96%, 97%, 98%, or 99% of the pores. The interconnected pores provide an increase in the osteoconductive properties of the device 131. The pores can be all the same size or may incorporate a gradient in pore size to accommodate the change in porosity in cortical and cancellous bone structure.

In some embodiments, the tapered scaffold structure 132 includes a mesh structure. The mesh structure may be composed of a stacking of bars to formulate the mesh. This style of meshing makes the tapered scaffold structure 132 easier to manufacture. In particular, the tapered scaffold structure 132 may be easily manufactured with fused deposition modeling.

FIG. 13C schematically shows a perspective view of the bone plug device 131 shown in FIG. 13A. In some embodiments, plug device 131 includes a solid rim 133 configured to help a user avoid pushing the device 131 through the cortical window. The tapered scaffold structure 132 may be combined with a tapered thread 137 to aid insertion of the device 131. The tapered thread 137 may be used to add interference with the cortex and create a tight fit. This configuration may assist in the retention of the device 131 within the cortical defect. In some embodiments, bone plug device 131 may include a female rim 133 that will pair with a male inserter that may be used to screw the device 131 into place.

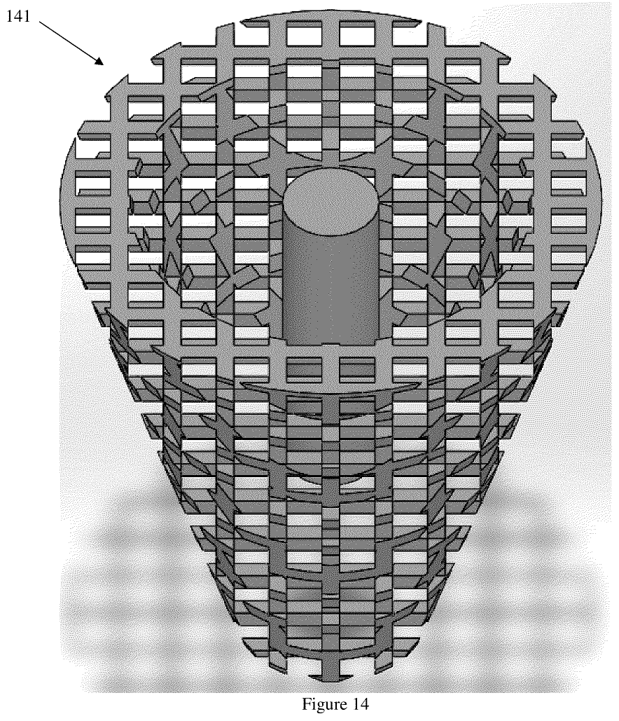

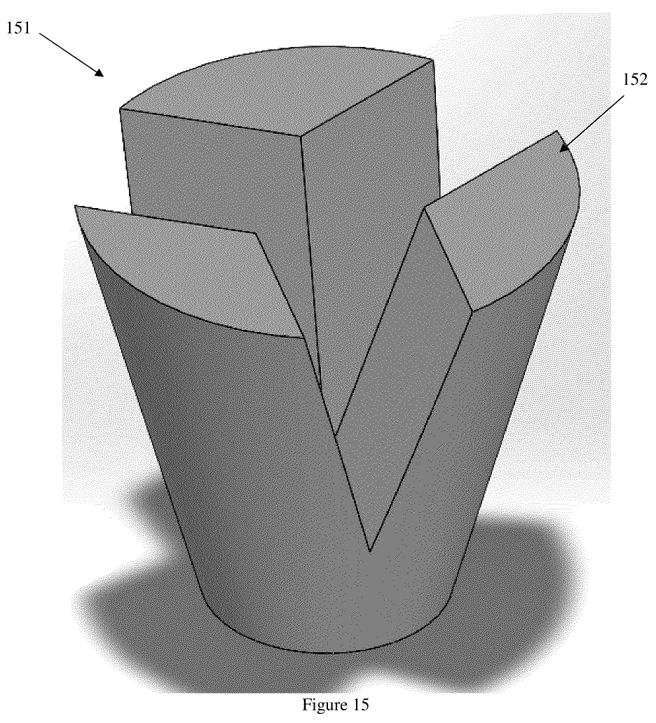

FIG. 14 schematically shows a perspective view of a tapered bone plug device 141 according to the present disclosure. The tapered plug device 141 may be filled with cancellous chips before placement in the cortical defect.

FIG. 15 schematically shows a perspective view of a three leaflet feature 152 of a bone plug device 151 according to the present disclosure. In some embodiments, the three leaflet feature 152 is structured such that each leaf will have more cortical contact as compared to a leaflet feature having four leaves. The three leaflet feature 152 can be combined with a rim, such as rim described above, to prevent the device 151 from falling through into a void when inserting the device 151 into the cortical hole.

Exemplary methods for fabrication of the bone plug devices of the present disclosure may generally include the following methods: (i) fused deposition modeling of a resorbable polymer, ceramic, mineral or other biocompatible material (ii) selective laser sintering (iii) injection molding (iv) machining, and the like.

A bone plug device as disclosed herein can include a mesh structure and a first and second solid structure. The mesh structure may define a proximal end and a distal end of the device. The mesh structure may include at least a first leaflet and a second leaflet joined at the distal end, and the mesh structure may be configured to mimic cancellous bone structure and porosity to encourage cancellous bone ingrowth. The first solid structure may be mounted or formed with respect to a proximal end of the leaflet. The second solid structure may be mounted or formed with respect to a proximal end of the second leaflet. The mesh structure may be moveable between (i) an open configuration in which the mesh structure defines a first diameter and in which the first solid structure is spaced from the second solid structure, and (ii) a closed configuration in which the mesh structure defines a second diameter smaller than the first diameter and in which the first solid structure contacts the second solid structure. The second diameter can conform to a diameter of a hole in a cortical bone.

A method of plugging a hole in cortical bone can include selecting a bone plug device including a mesh structure that defines a proximal end and a distal end of the device, the mesh structure including at least a first leaflet and a second leaflet joined at the distal end of the device, the mesh structure configured to mimic cancellous bone structure and porosity to encourage cancellous bone ingrowth; a first solid structure mounted or formed with respect to a proximal end of the first leaflet; and a second solid structure mounted or formed with respect to a proximal end of the second leaflet. The method can further include inserting the bone plug device into the hole in the cortical bone, wherein said insertion causes the mesh structure to move from (i) an open configuration in which the mesh structure defines a first diameter and in which the first solid structure is spaced from the second solid structure, to (ii) a closed configuration in which the mesh structure defines a second diameter smaller than the first diameter and in which the first solid structure contacts the second solid structure. The second diameter can conform to a diameter of the hole in a cortical bone.

In certain embodiments, a variable diameter or variable width bone plug device may comprise a mesh structure such as a tapered mesh structure that includes at least a first leaflet and a second leaflet that are joined together, for example, at a distal end or distal region of the mesh structure. The mesh structure is at least somewhat porous to encourage cancellous bone ingrowth. The bone plug device can also include a first less porous structure such as a solid structure that is joined to or otherwise located at a proximal end of the first leaflet. The first less porous structure is generally less porous than the mesh structure. The bone plug device can also include a second less porous structure such as a solid structure that is joined to or otherwise located at a proximal end of the second leaflet. The second less porous structure is generally less porous than the mesh structure. The mesh structure is biased toward an expanded first condition in a resting state yet is movable toward a compressed second condition in which the first less porous structure and the second less porous structure are moved closer to one another relative to the expanded first condition of the mesh structure.

In certain embodiments, a method of plugging a bone hole can comprise providing or obtaining a variable width or variable diameter bone plug device that is mounted on or otherwise coupled to an insertion device. The variable width bone plug device can comprise a mesh structure such as a tapered mesh structure that includes at least a first leaflet and a second leaflet that are joined together, for example, at a distal end or distal region of the mesh structure. The mesh structure is at least somewhat porous along all or part of its length to encourage cancellous bone ingrowth. The bone plug device can further include a first less porous structure such as a solid structure that is situated at a proximal end of the first leaflet and is typically but not necessarily less porous than the mesh structure. The bone plug device can further include a second less porous structure such as a solid structure that is situated at a proximal end of the second leaflet and is typically but not necessarily less porous than the mesh structure. Prior to the variable width bone plug device being mounted on the insertion device, the mesh structure can be biased toward an expanded first condition, for example, in a resting state. Thereafter, the mesh structure can be held in a compressed second condition when the variable width bone plug device is mounted on the insertion device. The compressed second condition can include the first less porous structure and the second less porous structure moved closer to one another relative to the expanded first condition of the tapered mesh structure. The method can further include inserting at least part of the bone plug device into the bone hole with the insertion device and separating the bone plug device from the insertion device to leave at least part of the mesh structure in the bone hole with the first leaflet and the second leaflet exerting outward force on bone surrounding the bone hole.

We claim:

1. A variable width bone plug device, comprising:
a mesh structure including at least a first leaflet and a second leaflet that are joined together at a distal end of the mesh structure, the mesh structure being porous to encourage cancellous bone ingrowth;
a first less porous structure located at a proximal end of the first leaflet and being less porous than the mesh structure; and
a second less porous structure located at a proximal end of the second leaflet and being less porous than the mesh structure,
wherein the mesh structure is biased toward an expanded first condition in a resting state of the mesh structure yet is movable toward a compressed second condition in which the first less porous structure and the second less porous structure are moved closer to one another relative to their positioning in the expanded first condition of the mesh structure.

2. The variable width bone plug device of claim 1, wherein the mesh structure is tapered in at least the resting state.

3. The variable width bone plug device of claim 1, wherein the first less porous structure and the second less porous structure contact one another in the compressed second condition.

4. The variable width bone plug device of claim 1, wherein at least one of the first less porous structure and the second less porous structure is non-porous.

5. The variable width bone plug device of claim 1, wherein the first less porous structure and the second less porous structure have a porosity to encourage cortical bone ingrowth.

6. The variable width bone plug device of claim 1, wherein the first less porous structure and the second less porous structure together form a rim in the compressed second condition.

7. The variable width bone plug device of claim 6, wherein the first less porous structure and the second less porous structure together form a flat rim in the compressed second condition.

8. The variable width bone plug device of claim 1, wherein the mesh structure includes an exterior protrusion.

9. The variable width bone plug device of claim 1, wherein the mesh structure includes an exterior barb.

10. The variable width bone plug device of claim 1, wherein the mesh structure includes an exterior threaded portion.

11. The variable width bone plug device of claim 1, wherein the mesh structure includes an interrupted exterior threading.

12. The variable width bone plug device of claim 1, wherein the mesh structure forms a series of successive rings formed at least in part by the first leaflet and the second leaflet.

13. The variable width bone plug device of claim 12, wherein the series of successive rings get progressively larger moving in a proximal direction along the mesh structure.

14. The variable width bone plug device of claim 1 further comprising a stem situated at least partially between the first leaflet and the second leaflet.

15. The variable width bone plug device of claim 1, wherein at least one of the first leaflet and the second leaflet includes a rail extending longitudinally along the respective leaflet.

16. The variable width bone plug device of claim 1 being cannulated.

17. A method of plugging a bone hole, comprising:
providing or obtaining a variable width bone plug device mounted on an insertion device, the variable width bone plug device comprising:
a tapered mesh structure including at least a first leaflet and a second leaflet that are joined together at a distal end of the tapered mesh structure, the tapered mesh structure being porous to encourage cancellous bone ingrowth;
a first less porous structure located at a proximal end of the first leaflet and being less porous than the tapered mesh structure; and
a second less porous structure located at a proximal end of the second leaflet and being less porous than the tapered mesh structure,
wherein, prior to the variable width bone plug device being mounted on the insertion device, the tapered mesh structure is biased toward an expanded first condition, and wherein the tapered mesh structure is held in a compressed second condition when the variable width bone plug device is mounted on the insertion device, the compressed second condition including the first less porous structure and the second less porous structure moved closer to one another relative to their positioning in the expanded first condition of the tapered mesh structure;
inserting at least part of the variable width bone plug device in the bone hole with the insertion device; and
separating the variable width bone plug device from the insertion device to leave at least part of the tapered mesh structure in the bone hole with the first leaflet and the second leaflet exerting outward force on bone surrounding the bone hole.

18. The method of claim 17, wherein said separating leaves the first less porous structure and the second less porous structure fully covering the bone hole.

19. The method of claim 17, wherein the tapered mesh structure in the compressed second condition is narrower than the bone hole.

20. The method of claim 17, wherein the tapered mesh structure in the compressed second condition is wider than the bone hole so that said inserting causes cortical bone surrounding the bone hole to further compress the tapered mesh structure during said inserting.
